(19) United States
(12) Patent Application Publication
(10) Pub. No.: US 2014/0088594 A1
(43) Pub. Date: Mar. 27, 2014

(54) INSTRUMENT FOR LOCATING DISTAL SCREW HOLES IN INTRAMEDULLARY NAILS

(71) Applicant: Jude L. Sasing, Diliman, Quezon City (PH)

(72) Inventor: Jude L. Sasing, Diliman, Quezon City (PH)

(73) Assignee: ORTHOPAEDIC INTERNATIONAL, INC., SEPZ, Cabuyao, Laguna (PH)

(21) Appl. No.: 14/007,394

(22) PCT Filed: Mar. 22, 2012
(86) PCT No.: PCT/PH12/00003

§ 371 Date: Dec. 9, 2013

Related U.S. Application Data
(60) Provisional application No. 61/467,614, filed on Mar. 25, 2011.

(51) Int. Cl. A61B 17/88; A61B 17/72

(52) U.S. Cl. CPC A61B 17/88; A61B 17/72
USPC 606/62

(57) ABSTRACT

A distal targeting device for locating at least one distal screw hole in an intramedullary nail that is positioned within a medullary canal of a bone, the device including an elongated and reconfigurable probe that is positionable inside a lumen of the nail.

### CROSS-REFERENCE TO RELATED APPLICATION

This application claims the benefit under 35 U.S.C. §119(e) of U.S. Provisional Patent Application No. 61/467,614, filed Mar. 25, 2011, which is incorporated herein by reference in its entirety.

### TECHNICAL FIELD

The present invention relates to locating the distal screw holes in intramedullary nails without using X-rays. In particular, the invention is used for locating distal screw holes by determining the anterior-posterior and/or the medial-lateral displacement of the distal screw holes and using this information to align an external targeting jig.

### BACKGROUND

Fractures of long bones are often treated with an intramedullary (IM) nail. An IM nail is a tubular metal implant bent a certain way to conform to the anatomy of the long bone being treated, such as the femur. An IM nail is inserted into the medullary canal of the bone and locked to the proximal and distal bone fragments with interlocking screws through holes located in the proximal and distal portions of the nail. Although locking both portions of the IM nail in place can be difficult because the nail is inside the bone and the screw holes cannot be visualized, locking the IM nail to the proximal portion of the bone can be somewhat easier to perform with the aid of targeting jigs attached to the proximal end of the nail, since the proximal portion is closer to the entry point of the nail into the bone than the distal portion. On the other hand, distal locking is more difficult because the nail is further from the entry point of the nail into the bone and targeting jigs attached to the proximal end of the nail can become misaligned with the distal holes when the nail deforms as it is inserted into the bone.

One common method of locating the distal screw holes is by using a fluoroscope or C-arm image intensifier to visualize the screw hole and target it from outside the bone with a hand-held drill. However, X-ray exposure from repeated use of a C-arm poses risks to surgeons who accumulate significant amounts of scattered X-ray radiation throughout their professional careers. In addition, C-arms are expensive and not commonly available in all hospitals.

Several targeting jigs have been proposed and created to locate the distal screw holes without the use of X-rays. However, many of these devices do not work consistently because of the misalignment that can be caused when the nail deforms. Thus, there is a need for improved devices and methods for locating the distal screw holes in an IM nail without the use of X-rays.

### SUMMARY

The devices and tools of the invention provide for accurate locating of distal screw holes in intramedullary nails. In a first configuration of the invention, a distal targeting device is provided for locating at least one distal screw hole in an intramedullary nail that is positioned within a medullary canal of a bone, the device comprising an elongated and reconfigurable probe that is positionable inside a lumen of the nail. The reconfigurable probe can include a plurality of segments extending longitudinally along a probe axis, wherein each of the plurality of segments is flexibly connected to at least one adjacent segment by a hinge. The plurality of segments of a particular probe can be rigid, flexible, or can include a combination of rigid and flexible segments, and the hinges can include a pivoting hinge or a flexible hinge. The plurality of segments can include one or more segments having ends with an associated extending member that is larger in at least one dimension than the segment end, but smaller than the inner lumen of an intramedullary nail in which it will be positioned.

The distal targeting device can further include an adjustable external jig that is adjustable in at least one of an anterior-posterior direction and a medial-lateral direction to correspond to a location of the at least one distal screw hole in response to information received from at least one data-gathering member of the probe.

### BRIEF DESCRIPTION OF THE DRAWINGS

The present invention will be further explained with reference to the appended Figures, wherein like structure is referred to by like numerals throughout the several views, and wherein:

FIG. 1 is a perspective view of a prior art intramedullary nail;

FIG. 2 is a perspective view of a measuring probe of the invention;

FIG. 3 is an enlarged perspective view of one of the flexible hinges of a measuring probe of the type illustrated in FIG. 2;

FIG. 4a is a detailed perspective view of a flexible hinge of a measuring probe, with a strain gage attached to the hinge;

FIG. 4b is a detailed perspective view of a flexible hinge of a measuring probe, with two strain gages attached to the hinge;

FIG. 5 is a perspective view of an embodiment of the invention including a measuring probe and an external jig;

FIG. 6 is a perspective view of an embodiment of the invention being used with an intramedullary nail;

FIG. 7 is a perspective view of another embodiment of a measuring probe of the invention;

FIG. 8 is an enlarged perspective view of a hinge of the measuring probe of FIG. 7;

FIG. 9 is a perspective view of another embodiment of a measuring probe of the invention;

FIG. 10 is a perspective view of another embodiment of an external jig, in accordance with the invention; and

FIG. 11 is a perspective view of the external jig of FIG. 10 and the measuring probe of FIG. 9 in use with an intramedullary nail.

### DETAILED DESCRIPTION

External targeting jigs that are currently being used to locate distal screw holes for intramedullary (IM) nails are often inadequate due to bending deformations of the nail as it is inserted into the bone. For most nailing applications, the most significant deformation of the nail that affects the accuracy of external jigs is the bending that occurs in the anterior-posterior direction. If the displacement of the screw holes in the anterior-posterior direction can be determined, the screw holes can be targeted accurately using an external jig. The devices and methods of the invention are used to measure the displacement of distal screw holes in the anterior-posterior direction by using a probe placed into the lumen of the IM nail. An adjustable external jig is then aligned with the distal screw holes using the probe measurements.

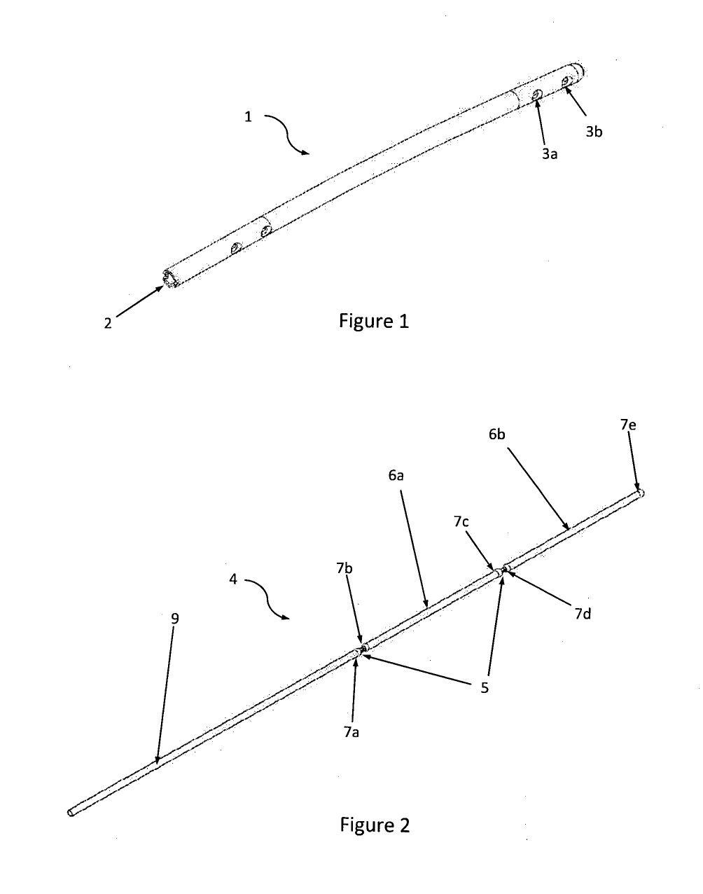

Referring now to the Figures, wherein the components are labeled with like numerals throughout the several Figures, and initially to FIG. 1, an exemplary prior art IM nail 1 with a lumen 2 going through its entire length and distal screw holes 3a and 3b is shown, which is one embodiment of an intramedullary nail. However, it is understood that other intramedullary nails having different configurations can also be used relative to the locating devices of the invention.

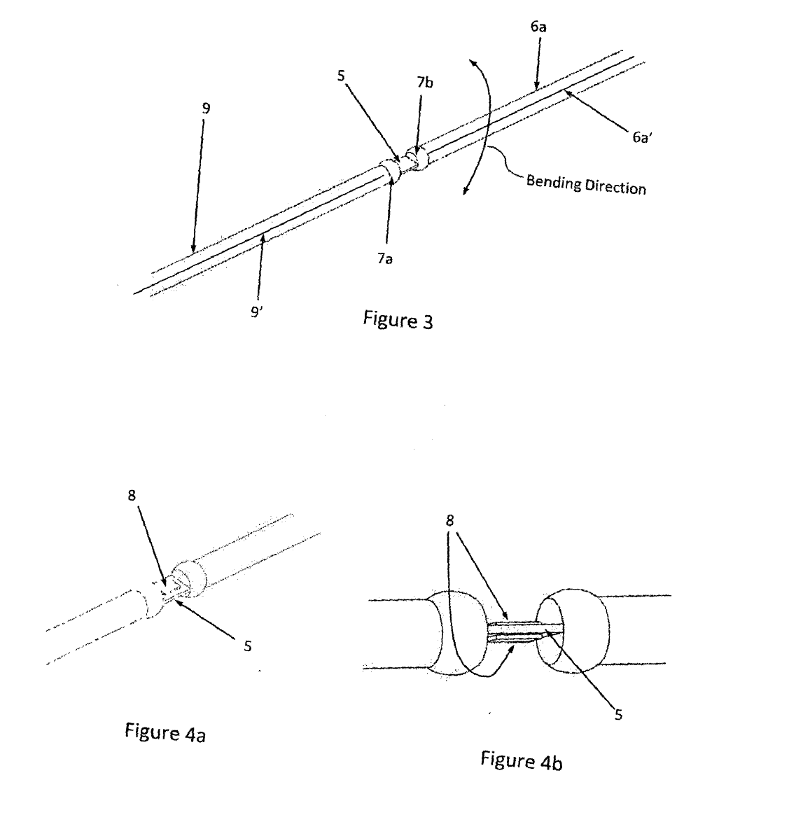

FIGS. 2 and 3 illustrate a measuring probe 4 of the invention, which includes a probe base 9, probe segments 6a and 6b, and two flexible hinges 5 between probe base 9 and segment 6a, and between segments 6a and 6b. While two hinges are shown and discussed herein, it is understood that a particular measuring probe can include more or less than two hinges. The flexible hinges 5 have a relatively thin cross-section, which may be rectangular, for example, and which allow bending in one plane, as shown in FIGS. 3, 4a, and 4b.

In operation, measuring probe 4 is inserted from the proximal end into lumen 2 of an IM nail, such as nail 1, such that the bending planes of flexible hinges 5 correspond to the bending plane of the nail (for example, parallel to the sagittal plane for femoral nails). As measuring probe 4 is inserted, the flexible hinges 5 can bend to allow the measuring probe 4 to conform to the bent or curved shape of lumen 2. Probe base 9 and probe segments 6a and 6b are relatively rigid compared to the flexible hinges 5. Due to the relative flexibility of flexible hinges 5 as compared to the adjacent areas of the probe structure, any deformation of measuring probe 4 as it is inserted into lumen 2 will be isolated and concentrated mainly or exclusively to the areas of the flexible hinges 5. The base end 7a of probe base 9 and segment ends 7b, 7c, 7d, and 7e of probe segments 6a and 6b are shown as spherical end portions having a diameter that closely matches the diameter of lumen 2 and which is larger than the diameter of the middle portions of probe base 9 and probe segments 6a and 6b. This configuration will allow probe 4 to deform in a relatively consistent manner since the contact points are limited to predetermined locations at base end 7a and segment ends 7b, 7c, 7d and 7e. The middle portions of probe base 9 and probe segments 6a and 6b can also be designed to have a bent or curved shape in order to better approximate the contour of lumen 2 and avoid contact with lumen 2 other than at the spherical ends.

It is noted that the use of the term “spherical” relative to “spherical end portions” or “spherical members” throughout the description is not intended to solely encompass an end portion shaped as an actual sphere. Rather, the use of the term spherical herein with regard to the end portions of the invention can instead have a different shape, such as elliptical, cubic, triangular and the like. In order to provide the advantages described herein relative to contact between these members and the inside of a lumen of an intramedullary nail, however, at least one of the dimensions of the “spherical” members at the ends of probe segments should be larger than at least one dimension of the outer surface of the corresponding probe segment. Thus, these spherical members are alternatively referred to herein as “extending members.”

FIGS. 4a and 4b illustrate exemplary embodiments of flexible hinge 5 in more detail. In the embodiment of FIG. 4a, a strain gage 8 is shown attached to one side surface of flexible hinge 5. Optionally, two strain gages can be attached, one on each side of flexible hinge 5, as shown in FIG. 4b. As measuring probe 4 deforms about one or more of the hinges 5 while being inserted into lumen 2, the amount of bending deformation at each flexible hinge 5 is measured by strain gage 8. As long as the bending stresses on flexible hinge 5 are kept within the elastic range, this bending deformation will have a linear correlation with the angular displacement of each segment of probe 4 relative to the adjacent segment, such as angular displacement of probe segment 6a relative to probe base 9. Multiplying the angular displacement by the length of the segment allows calculation of the displacement of each segment end relative to the axis of the adjacent segment, such as the displacement of segment end 7c relative to axis 9′, where axis 9′ is shown in FIG. 3. The displacement of segment end 7b relative to segment end 7a can be neglected due to their close proximity to each other, or a certain correction factor can be factored into the computations. Similarly, the displacement of segment end 7e relative to axis 6a′ (see FIGS. 2 and 3) can be computed from the angular displacement between segments 6a and 6b. Adding these displacements together will allow the calculation of the total displacement of the segment end 7e relative to base 9 of measuring probe 4. In operation, segment end 7e can be positioned near either of distal holes 3a or 3b so that the position of segment end 7e will closely correspond to the location of the distal hole relative to probe base 9.

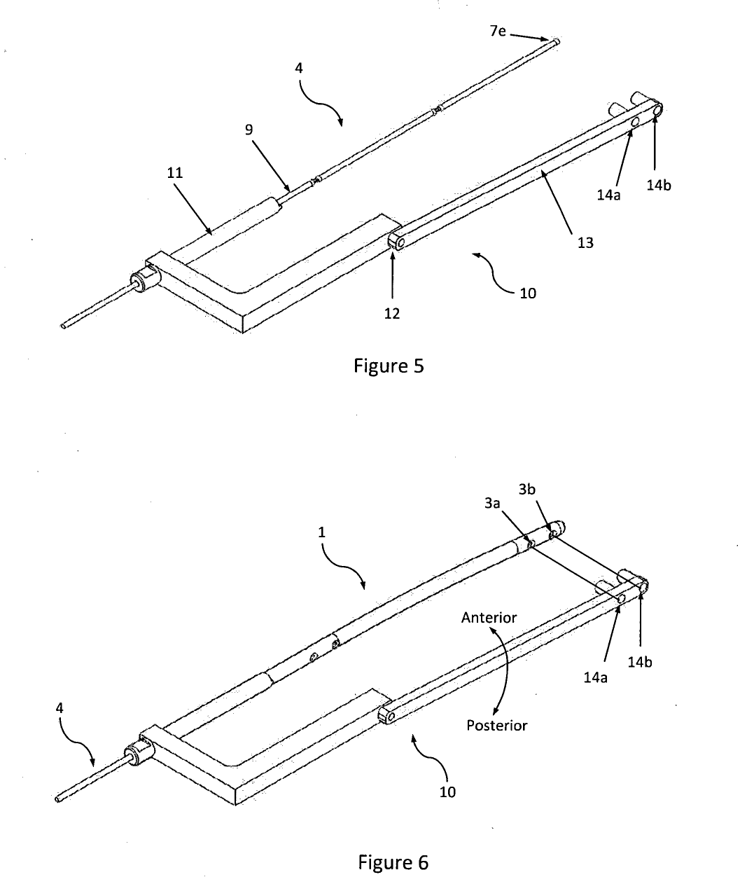

FIG. 5 illustrates base 9 attached to an external jig 10. External jig 10 has a nail attachment portion 11, which can be rigidly attached to the proximal end of nail 1, a pivoting joint 12, and an extension arm 13 with guide holes 14a and 14b. Extension arm 13 is calibrated in such a way that the distance of the guide holes 14a and 14b from the proximal end of nail 1, when measured along the axis of the nail, corresponds to the distance of the distal holes 3a and 3b from the proximal end of nail 1, respectively, when measured along the nail axis. Using the known position of segment end 7e, as can be computed from the strain gage readings as discussed above, the user will be able to position extension arm 12 in the anterior-posterior direction such that guide holes 14a and 14b will be aligned with the distal holes 3a and 3b of intramedullary nail 1 respectively, as is shown in FIG. 6.

Any or all of the strain gages 8 can be connected to appropriate electronic circuitry and devices to measure the strains at flexible hinges 5. The strain values in turn can be converted to displacement data by calibration or by using appropriate equations and conversion factors known to those skilled in the art. Although not illustrated in the figures, spaces for electrical wiring to the strain gages can be made, for example, by hollowing out or cutting grooves along the lengths of segments 6a and 6b, and/or of base 9.

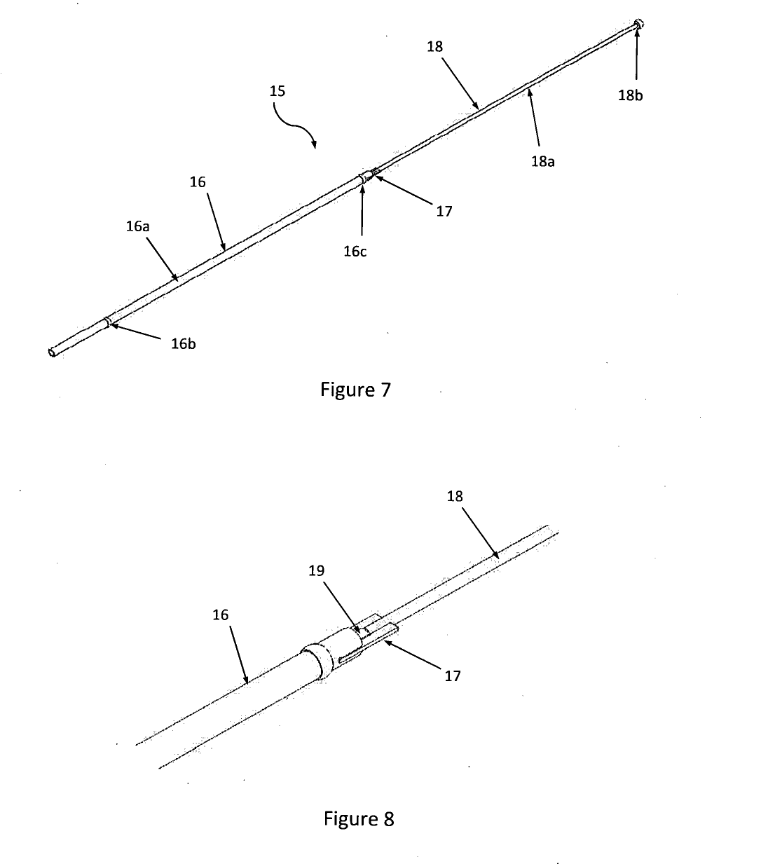

FIG. 7 illustrates another embodiment of a measuring probe 15 of the invention. In this embodiment, rather than having rigid segments connected by flexible hinges discussed relative to the above embodiment, FIG. 7 provides for a probe 15 composed of a rigid base member 16, a flexible hinge 17, and a flexible segment 18. Base member 16 is composed of a roughly cylindrical portion 16a, along with two spherical portions 16b and 16c having diameters that closely match the diameter of lumen 2 of the intramedullary nail 1 in which the probe 15 will be positioned. The diameters of the spherical portions 16b and 16c can also be larger than the diameter of cylindrical portion 16a. Flexible hinge 17 is attached to base 16 and also to flexible segment 18, as shown in FIG. 8. Flexible hinge 17 has a thin rectangular cross section and can also be equipped with one or more strain gages, such as strain gage 19. Flexible segment 18 is composed of a flexible portion 18a and a spherical portion 18b at its distal end, where the spherical portion 18b can have a diameter that closely matches the diameter of lumen 2 of nail 1, and which is larger than the diameter of flexible portion 18a. Flexible portion 18a is constructed to be much more flexible than rigid base member 16, such that when subjected to the same bending moment, the deformation of rigid base member 16 will be significantly less than the deformation of flexible portion 18a.

As probe 15 is inserted into the intramedullary nail 1, the portion of probe 15 comprising flexible hinge 17 and flexible segment 18 behaves like a cantilever beam and bends to approximate the contour of the distal part of nail 1, while rigid base 16 remains substantially straight, thereby approximating the straight contour of nail attachment portion 11 and the proximal part of nail 1. Thus, contact between probe 15 and the inner walls of nail attachment portion 11 and lumen 2 is limited mainly or exclusively to spherical portions 16b, 16c, and 18b. Measurements from strain gage 19 will be directly proportional to the displacement of spherical portion 18b relative to base 16 and can be used to locate distal holes 3a and 3b of intramedullary nail 1. Flexible portion 18a can also be designed to relatively closely approximate the nail contour when it bends, for example, by having a tapering cross section instead of a constant cylindrical cross section along its length, or by having it pre-bent in a certain way.

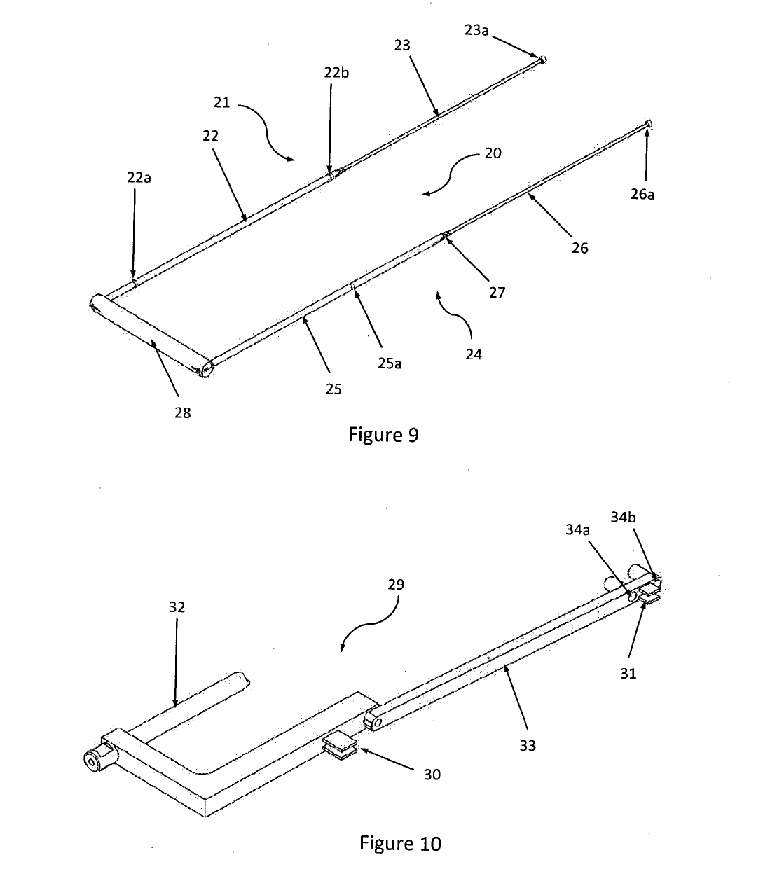

FIG. 9 illustrates another exemplary embodiment of a measuring probe of the invention. As is illustrated in this Figure, a probe assembly 20 is composed of two probes 21 and 24 that can have substantially similar constructions to each other, and which are rigidly connected to each other via a connector member 28. Probes 21 and 24 can be generally constructed as described above relative to the construction of probe 15, except that base 25 of probe 24 has only one spherical portion 25a as compared to the two spherical portions 22a and 22b of base 22 of probe 21. This is intended to avoid redundant supports and provide for a stable and repeatable orientation of base 22 and base 25 relative to an exemplary external jig 29 that is shown in FIG. 10.

Since probes 21 and 24 are substantially similar in construction, and contact to the probes when the probe assembly 20 is in use is limited mainly or exclusively to spherical portions 22a, 22b, 23a, 25a, and 26a, the positions of spherical portions 23a and 26a in the anterior-posterior direction relative to base 22 and base 25 will be the same if the strain gage readings for probes 21 and 24 are the same. A calibration factor may be used to accommodate any variations between probes 21 and 24.

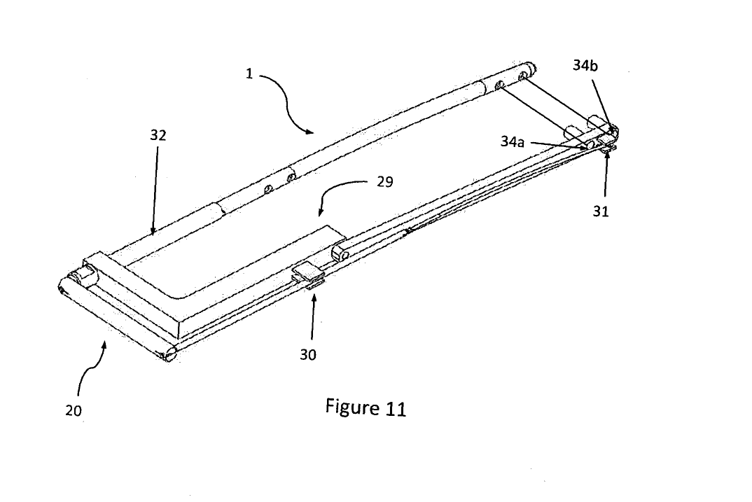

FIG. 10 illustrates an external jig 29 and FIG. 11 illustrates nail 1 attached to the external jig 29, with probe assembly 20 inserted into nail 1 and external jig 29. External jig 29 is equipped with guide walls 30 and 31 to simulate the inner wall of nail attachment portion 32 and lumen 2 of nail 1, and to provide contact points with spherical portions 25a and 26a. In operation, probe 21 is inserted into nail 1 such that spherical portion 23a is at or near the distal hole to be targeted, while probe 24 is inserted through the guide walls 30 and 31. Extension arm 33 is then adjusted in the anterior-posterior direction, while bending flexible segment 26 and flexible hinge 27 of probe 24 in the process, until the strain gage readings in probes 21 and 24 are identical. At this point, the positions of spherical portions 23a and 26a relative to base 22 and base 25, respectively, will also be identical. Thus, guide hole 34a (and/or guide hole 34b) will be aligned with the corresponding nail distal hole being targeted.

In accordance with the invention described herein, any of the deformable probes can be designed to provide for contact with the inner lumen of an intramedullary nail and/or nail holding instrument, and can be limited to a certain number of predetermined points. The purpose of this is to ensure repeatability and accuracy of measurements. If contact points are not accurately known, the readings received from strain gages will not be repeatable. In other words, if the contact points differ, the readings for the same position of the distal end of the probe can be different.

The concepts described above can also be modified by using more or less flexible hinges (and corresponding number of segments) than are illustrated in the figures, rigid segments, and flexible segments, if desired, such for the purpose of accommodating sharper or shallower bending of the intramedullary nail, for example. The hinges can also be designed to allow bending in more than one plane to accommodate bending deformations in more than a single plane.

The present invention has now been described with reference to several embodiments thereof. The entire disclosure of any patent or patent application identified herein is hereby incorporated by reference. The foregoing detailed description and examples have been given for clarity of understanding only. No unnecessary limitations are to be understood therefrom. It will be apparent to those skilled in the art that many changes can be made in the embodiments described without departing from the scope of the invention. Thus, the scope of the present invention should not be limited to the structures described herein, but only by the structures described by the language of the claims and the equivalents of those structures.

1. A distal targeting device for locating at least one distal screw hole in an intramedullary nail that is positioned within a medullary canal of a bone, the distal targeting device comprising an elongated and deformable probe that is positionable inside a lumen of the nail, the probe comprising:
a base;
at least one segment extending in a longitudinal direction of the base, wherein at least one of the segments comprises two opposing ends;
at least one extending member extending beyond an outer surface of at least one of the two opposing ends of the at least one segment, wherein the extending member is configured for contact with inner surfaces of the lumen of the nail; and
at least one hinge operatively engaged with at least one of the outer surfaces of the thwo opposing ends of the at least one segment, wherein the at least one hinge has a means for measuring the deformation of the probe;
wherein the at least one extending member comprises at least one pre-determined contact point between the probe and the inner surfaces of the lumen of the nail, whereby the at least one pre-determined contact point provides for axial and rotational movements of the probe relative to the nail, thereby enabling the probe to accurately follow the contour of the lumen of the nail and to deform in a substantially consistent manner to provide for repeatability and accuracy in the measurement of the deformation of the probe.

2. (canceled)

3. The distal targeting device of claim 1, wherein at least one of the segments is rigid.

4. The distal targeting device of claim 1, wherein at least one of the segments is flexible.

5. (canceled)

6. The distal targeting device of claim 1, wherein at least one of the hinges is flexible.

7. The distal targeting device of claim 1, wherein the means fof measuring the deformation of the probe is arranged to measure the displacement of at least two segments relative to each other.

8. The distal targeting device of claim 1, wherein the means of measuring the deformation of the probe comprises at least one strain gauge.

9. (canceled)

10. The distal targeting device of claim 1, further comprising an adjustable external jig that is adjustable in at least one of an anterior-posterior direction and a medial-lateral direction to correspond to a location of the at least one distal screw hole in response to information received from the means for measuring the deformation of the probe.

11. The distal targeting device of claim 10, wherein a correlation of a position of the jig in relation to the information received from the means for measuring the deformation of the probe is achieved using at least one of a mechanical displacement scale and an electronic displacement scale located on the jig.

12. The distal targeting device of claim 10, wherein a correlation of a position of the jig in relation to the information received from the means for measuring the deformation of the probe is achieved using an external probe that is substantially identical to the internal probe, wherein the external probe is coupled to the external jig so that a first jig position is indicated by a concurrence of readings between the internal and external probes.

13. (canceled)

14. (canceled)

15. The distal targeting device of claim 1, wherein at least one segment has comprises an outer diameter, and wherein the at least one first extending member has an outer diameter that is larger than the outer diameter of the at least one segment.

16. (canceled)

17. (canceled)

18. (canceled)

19. The distal targeting device of claim 1, wherein the at least one segment comprises a plurality of segments extending in a longitudinal direction of the base, wherein each of the plurality of segments has two opposing ends, wherein the at least one hinge comprises a plurality of hinges, and wherein each of the plurality of hinges is adapted to flexibly connect at least two of the plurality of segments.

20. The distal targeting device of claim 19, wherein at least one of the two opposing ends of each of the plurality of segments is provided with an extending member extending beyond an outer surface thereof, and wherein the extending member is arranged to be in contact with the inner surfaces of the lumen of the nail.

21. The distal targeting device of claim 20, wherein a plurality of pre-determined contact points are formed by the extending member associated with each of the plurality of segments, whereby the plurality of pre-determined contact points enables the probe to deform in a substantially consistent manner to provide for repeatability and accuracy in the measurement of the deformation of the probe.

22. The distal targeting device of claim 1, wherein the means for measuring the deformation of the probe is arranged to measure the displacement of the at least one segment relative to the base.

23. The distal targeting device of claim 1, wherein the at least one segment comprises a single segment, wherein the single segment has two opposing ends, wherein the at least one hinge comprises a single hinge, and wherein the single hinge is disposed between the base and the at least one of the two opposing ends of the single segment.

24. The distal targeting device of claim 11, whereby measurement associated with the deformation of the probe is directly proportional to the displacement of the extending member relative to the base, to provide for locating the distal screw holes in the nail.

25. The distal targeting device of claim 23, wherein the means for measuring the deformation of the probe is arranged to measure the displacement of the extending member of the single segment relative to the base.

26. The distal targeting devices of claim 1, wherein the at least one pre-determined contact point is a portion of a sphere.
